SEQUENCE LISTING (21 sequences)

SEQ ID NO: 1 - skipped

SEQ ID NO: 2 - protein, 5 aa, synthetic construct
  REGION 1..5: note = 5GS linker
GGGGS

SEQ ID NO: 3 - protein, 7 aa, synthetic construct
  REGION 1..7: note = 7GS linker
SGGSGGS

SEQ ID NO: 4 - protein, 8 aa, synthetic construct
  REGION 1..8: note = 8GS linker
GGGGSGGS

SEQ ID NO: 5 - protein, 9 aa, synthetic construct
  REGION 1..9: note = 9GS linker
GGGGSGGGS

SEQ ID NO: 6 - protein, 10 aa, synthetic construct
  REGION 1..10: note = 10GS linker
GGGGSGGGGS

SEQ ID NO: 7 - protein, 15 aa, synthetic construct
  REGION 1..15: note = 15GS linker
GGGGSGGGGSGGGGS

SEQ ID NO: 8 - protein, 18 aa, synthetic construct
  REGION 1..18: note = 18GS linker
GGGGSGGGGSGGGGSGGS

SEQ ID NO: 9 - protein, 20 aa, synthetic construct
  REGION 1..20: note = 20GS linker
GGGGSGGGGSGGGGSGGGGS

SEQ ID NO: 10 - protein, 25 aa, synthetic construct
  REGION 1..25: note = 25GS linker
GGGGSGGGGSGGGGSGGGGSGGGGS

SEQ ID NO: 11 - protein, 30 aa, synthetic construct
  REGION 1..30: note = 30GS linker
GGGGSGGGGSGGGGSGGGGSGGGGSGGGGS

SEQ ID NO: 12 - protein, 35 aa, synthetic construct
  REGION 1..35: note = 35GS linker
GGGGSGGGGSGGGGSGGGGSGGGGSGGGGSGGGGS

SEQ ID NO: 13 - protein, 40 aa, synthetic construct
  REGION 1..40: note = 40GS linker
GGGGSGGGGSGGGGSGGGGSGGGGSGGGGSGGGGSGGGGS

SEQ ID NO: 14 - protein, 15 aa, synthetic construct
  REGION 1..15: note = G1 hinge
EPKSCDKTHTCPPCP

SEQ ID NO: 15 - protein, 24 aa, synthetic construct
  REGION 1..24: note = 9GS-G1 hinge
GGGGSGGGSEPKSCDKTHTCPPCP

SEQ ID NO: 16 - protein, 12 aa, synthetic construct
  REGION 1..12: note = Llama upper long hinge region
EPKTPKPQPAAA

SEQ ID NO: 17 - protein, 62 aa, synthetic construct
  REGION 1..62: note = G3 hinge
ELKTPLGDTTHTCPRCPEPKSCDTPPPCPRCPEPKSCDTPPPCPRCPEPKSCDTPPPCPRCP

SEQ ID NO: 18 - DNA, 1728 bp, synthetic construct
  misc_feature 1..1728: note = Multivalent ISV
  source 1..1728: mol_type = other DNA
gacgtgcaattggtggagtctggtggcggagtggtgcagcctggaggatctctaagactctcctgtgcggcttctgggtttacgtttagctcgtttgccatgacttgggtgcgccgcccgccgggtaaaggcttggagtgggtggcgacgattacaaatgctggggttacgagttacgcggactcggtaaaaggccgcttcacaatctcgcgtgataatgcgaagaacacgttatatcttcagatgaacagccttcgcccggaggataccgcactctacatctgtgctaatgctcgtcgcactggccctcgtgcacccaccgacatcggaagctaccgcggacaaggaaccctggtcacggtctcctccggaggcggtggctcaggtggcggaggcagcggtggaggaggtagtgggggtggaggcagcggcggtggcggtagtggaggcggaggcagtgggggcggtggatccgaggtgcagttggtggagtctgggggaggcttggtgcaggctggggggtctctgagactctcctgtgcagcctctggaagtattgtcggcatccatagtgtgggctggtatcgccaggttcctgggaggcagcgcgagttggtcgcaactattgtcactgatactgggacgcactatcgagacttcgtgaagggccgattcaccatctccagagacatcgccaagaatacattgtaccttcaaatgaatagccttcagcctgaggacacagccgtctattattgttatttcaatctaatacgcgggcgctactggggccaggggaccctggtcacggtctcgagcggaggcggtgggtcaggtggaggaggtagtggtggcggaggaagcggcggtggcggtagtgggggtggaggcagcggaggcggtggctctggaggcggtggctcagaggtacaactagtggagtctggtggtggtgtggtgcagccgggtggttctttacgtctgagctgtgccgccagcggtagcatctttaccagcgccgtgatggaatggtttcgtcaagctccgggtaaggaacgcgtgctggttgcccgtattggtagcggtggtcgcattgcctatgtggatagcgtgaaaggtcgcttcaccatcagccgcgataacagcaagaacaccgtgactttacagatgaactctttacgcccggaagataccgctttatattattgccgcagcgtgattggtggctacatttattggggccaaggtaccctggtcacagtctcctcaggtggcggtggctcaggtggaggaggtagtggtggaggaggtagtgggggtggaggcagcggcggtggcggtagtggaggcggtggctccggaggcggtggctcagaggtacagttggtggagtctggtggaggcgtagtccaacccggtggtagtctgcgccttagctgcgctgcaagtggattcacgtttaggtcttttggtatgagttgggtcagacaggctcccggcaagggaccagagtgggtctcctcaatttctggatctggatcagatacattgtacgcagactctgtgaaaggtaggttcacgatttctagggacaactcaaagaatacgctgtacttacaaatgaactcattacgtcccgaggatacagccttgtattattgcacaatcggcggcagtctatccagatcatcccaaggaaccctggtcaccgtctcctcagcgtgataa

SEQ ID NO: 19 - DNA, 1638 bp, synthetic construct
  misc_feature 1..1638: note = Multivalent ISV
  source 1..1638: mol_type = other DNA
gacgtgcaattggtggagtctggtggcggagtggtgcagcctggaggatctctaagactctcctgtgcggcttctgggtttacgtttagctcgtttgccatgacttgggtgcgccgcccgccgggtaaaggcttggagtgggtggcgacgattacaaatgctggggttacgagttacgcggactcggtaaaaggccgcttcacaatctcgcgtgataatgcgaagaacacgttatatcttcagatgaacagccttcgcccggaggataccgcactctacatctgtgctaatgctcgtcgcactggccctcgtgcacccaccgacatcggaagctaccgcggacaaggaaccctggtcacggtctcctccggaggcggtggctcaggcggtggcggtagtggaggcggaggcagtgggggcggtggatccgagggsarwtggtkgrgtctgggasggkcycgakcggaggcggkgggttaggtggwrsasgkagtggrggcrrtgsckctgcasrcggtsgckyartrrtrcgactagtasrgtctggckkcagdgkagcagcgcysgstggtwgkatacstcwswsctgatcygygmdcgkkctcrtcdyactcrgygycgtgcwgsmacggwytcskcaagctccsggkvasgaasacgtsytgkctscsyvtmytcgkrrsgckgkbsgyvwkgcckwggyggtartsgtgrmrgakscaktsrsgrcggkggatccgargggagwtggyggmgtcwgtcacggtctcgagcggaggcggtgggtcaggtggaggaggtagtggaggcggtggctctggaggcggtggctcagaggtacaactagtggagtctggtggtggtgtggtgcagccgggtggttctttacgtctgagctgtgccgccagcggtagcatctttaccagcgccgtgatggaatggtttcgtcaagctccgggtaaggaacgcgtgctggttgcccgtattggtagcggtggtcgcattgcctatgtggatagcgtgaaaggtcgcttcaccatcagccgcgataacagcaagaacaccgtgactttacagatgaactctttacgcccggaagataccgctttatattattgccgcagcgtgattggtggctacatttattggggccaaggtaccctggtcacagtctcctcaggtggcggtggctcaggtggaggaggtagtggtggaggaggtagtgggggtggaggcagcggcggtggcggtagtggaggcggtggctccggaggcggtggctcagaggtacagttggtggagtctggtggaggcgtagtccaacccggtggtagtctgcgccttagctgcgctgcaagtggattcacgtttaggtcttttggtatgagttgggtcagacaggctcccggcaagggaccagagtgggtctcctcaatttctggatctggatcagatacattgtacgcagactctgtgaaaggtaggttcacgatttctagggacaactcaaagaatacgctgtacttacaaatgaactcattacgtcccgaggatacagccttgtattattgcacaatcggcggcagtctatccagatcatcccaaggaaccctggtcaccgtctcctcagcgtgataa

SEQ ID NO: 20 - protein, 574 aa, synthetic construct
  REGION 1..574: note = Multivalent ISV
DVQLVESGGGVVQPGGSLRLSCAASGFTFSSFAMTWVRRPPGKGLEWVATITNAGVTSYADSVKGRFTISRDNAKNTLYLQMNSLRPEDTALYICANARRTGPRAPTDIGSYRGQGTLVTVSSGGGGSGGGGSGGGGSGGGGSGGGGSGGGGSGGGGSEVQLVESGGGLVQAGGSLRLSCAASGSIVGIHSVGWYRQVPGRQRELVATIVTDTGTHYRDFVKGRFTISRDIAKNTLYLQMNSLQPEDTAVYYCYFNLIRGRYWGQGTLVTVSSGGGGSGGGGSGGGGSGGGGSGGGGSGGGGSGGGGSEVQLVESGGGVVQPGGSLRLSCAASGSIFTSAVMEWFRQAPGKERVLVARIGSGGRIAYVDSVKGRFTISRDNSKNTVTLQMNSLRPEDTALYYCRSVIGGYIYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 21 - protein, 541 aa, synthetic construct
  REGION 1..541: note = Multivalent ISV
  VARIANT 146: note = X can be any amino acid
  VARIANT 148..149: note = X can be any amino acid
  VARIANT 152..153: note = X can be any amino acid
  VARIANT 155: note = X can be any amino acid
  VARIANT 158: note = X can be any amino acid
  VARIANT 161..163: note = X can be any amino acid
  VARIANT 165: note = X can be any amino acid
  VARIANT 167..168: note = X can be any amino acid
  VARIANT 170..171: note = X can be any amino acid
  VARIANT 174..176: note = X can be any amino acid
  VARIANT 178..179: note = X can be any amino acid
  VARIANT 182..184: note = X can be any amino acid
  VARIANT 187..188: note = X can be any amino acid
  VARIANT 190: note = X can be any amino acid
  VARIANT 192..194: note = X can be any amino acid
  VARIANT 196..199: note = X can be any amino acid
  VARIANT 202: note = X can be any amino acid
  VARIANT 204..205: note = X can be any amino acid
  VARIANT 207..208: note = X can be any amino acid
  VARIANT 210..211: note = X can be any amino acid
  VARIANT 214..217: note = X can be any amino acid
  VARIANT 219..220: note = X can be any amino acid
  VARIANT 222..230: note = X can be any amino acid
  VARIANT 232..240: note = X can be any amino acid
  VARIANT 242: note = X can be any amino acid
  VARIANT 247..249: note = X can be any amino acid
  VARIANT 245: note = X can be any amino acid
DVQLVESGGGVVQPGGSLRLSCAASGFTFSSFAMTWVRRPPGKGLEWVATITNAGVTSYADSVKGRFTISRDNAKNTLYLQMNSLRPEDTALYICANARRTGPRAPTDIGSYRGQGTLVTVSSGGGGSGGGGSGGGGSGGGGSEGXWXXLGXXRXEAXGVXXXVXAXXLXXVAXXXDXXLAXXXQRXXVXYXXXDXXXXSSXSXXCXXGXXKLXXXXTXXLXXXXXXXXXPXXXXXXXXXRXIRXEXXXSVTVSSGGGGSGGGGSGGGGSGGGGSEVQLVESGGGVVQPGGSLRLSCAASGSIFTSAVMEWFRQAPGKERVLVARIGSGGRIAYVDSVKGRFTISRDNSKNTVTLQMNSLRPEDTALYYCRSVIGGYIYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

(19) United States
(12) Patent Application Publication
(10) Pub. No.: US 2026/0045320 A1
(43) Pub. Date: Feb. 12, 2026

(54) Obtaining Sequence Information For Target Multivalent Immunoglobulin Single Variable Domains

(71) Applicant: Ablynx N.V., Gent (BE)

(72) Inventors: Yanik Bruynooghe, Gent (BE); Norbert Furtmann, Frankfurt am Main (DE); Raf Ponsaerts, Gent (BE)

(21) Appl. No.: 18/709,712

(22) PCT Filed: Nov. 22, 2022
(86) PCT No.: PCT/EP2022/082767

§ 371 Date: May 13, 2024

(30) Foreign Application Priority Data
Nov. 22, 2021 (EP) 21209696.0

(51) Int. Cl. G16B 35/00; G16B 15/30

(52) U.S. Cl. CPC G16B 35/00; G16B 15/30

(57) ABSTRACT

A computer-implemented method for obtaining sequence information for each of a plurality of target multivalent immunoglobulin single variable domains (ISVs) comprises: receiving sequence information for component ISVs; generating candidate sequences of multivalent ISVs based on the received sequence information; obtaining groups of reads of sequencing information corresponding to target multivalent ISVs; for each read: determining hit candidate sequences from the set of candidate sequences and generating consensus matrices for hit candidate sequences; generating, for each group of reads, an assembly matrix for each hit candidate sequence; and determining sequence information for each target multivalent ISV.

### CROSS REFERENCE TO RELATED APPLICATIONS

The present application is the national stage entry of International Patent Application No. PCT/EP2022/082767, filed on Nov. 22, 2022, and claims priority to Application No. EP 21209696.0, filed on Nov. 22, 2021, the disclosures of which are incorporated herein by reference.

### TECHNICAL FIELD

This specification relates to obtaining sequence information for target multivalent immunoglobulin single variable domains (ISVs) based on received sequence information for a plurality of component ISVs.

### BACKGROUND

Obtaining sequence information, e.g. a DNA sequence, for multivalent ISVs is a difficult task. For example, the sequence information for a multivalent immunoglobulin single variable domain (ISV) is normally too large to be sequenced at once using conventional sequencing techniques. Techniques that sequence fragments of a multivalent ISV to obtain a sequence of the entire multivalent ISV require joining together sequence information (or portions thereof) for each of the fragments, which is a time-consuming and difficult task as, for example, many repetitive sequences may be present in each of the sequenced fragments.

### SUMMARY

According to a first aspect of this specification, there is described a computer-implemented method for obtaining sequence information for each of a plurality of target multivalent immunoglobulin single variable domains (ISVs). The method comprises: receiving sequence information for each of a plurality of component ISVs, wherein each target multivalent immunoglobulin single variable domain (ISV) comprises a plurality of the component ISVs; generating a set of candidate sequences of multivalent ISVs based on the received sequence information; obtaining a plurality of groups of reads of sequencing information, wherein each group of reads corresponds to a particular target multivalent ISV of the plurality of target multivalent ISVs; for each read of a group of reads: determining one or more hit candidate sequences from the set of candidate sequences, wherein each of the one or more hit candidate sequences comprises a matching portion with a corresponding portion of the read, and generating a consensus matrix for each hit candidate sequence using the hit candidate sequence, the read, and one or more sequences derived from the read, wherein the consensus matrix specifies, for each position of a plurality of positions in an alignment sequence, a consensus between the hit candidate sequence, the read, and the one or more sequences derived from the read, generating, for each group of reads, an assembly matrix for each hit candidate sequence based on the consensus matrix of each read in the group of reads; and determining sequence information for each target multivalent ISV based on one or more assembly matrices determined for the group of reads corresponding to the target multivalent ISV.

This can allow determination of sequence information for each of a plurality of target multivalent ISVs in an automated and rapid manner, e.g. determining sequence information for 96 clones in a few minutes.

A read may comprise a letter code for each position of a plurality of positions of the read. Each letter code may specify either a letter code for a primary base or an ambiguity letter code. Determining, for a read, one or more hit candidate sequences from the set of candidate sequences may comprise removing one or more letter codes from the end of the read to produce a shortened read for each iteration of a plurality of iterations. The determining may further comprise performing a pattern matching process between the shortened read of an iteration and each candidate sequence. The determining may further comprise: when a shortened read of an iteration matches a particular candidate sequence, adding the particular candidate sequence to the one or more hit candidate sequences.

This can allow determination of sequence information for each target multivalent ISV despite e.g. errors or mistakes in sequencing; removing a small number of letter codes at a time leads to fewer hit candidate sequences at each iteration and therefore provide a greater likelihood to determine accurate sequence information for the target multivalent ISVs.

A read may specify a sequencing quality for each position. Determining, for a read, one or more hit candidate sequences from the set of candidate sequences may comprise receiving a cutoff parameter; determining a trimmed read, comprising removing one or more letter codes of the read that each has a sequencing quality lower than a value specified by the cut-off parameter; determining a start position for the read, and removing letter codes of the read before the start position; determining a position of the read that first specifies an ambiguity letter code; and removing letter codes of the read that have a position beginning from the determined position until an end position of the read.

This can help to ensure that the reads are of sufficiently high quality.

Each candidate sequence in the hit candidate sequences may comprise a respective matching portion corresponding to each read in the group of reads.

This can help to ensure that the hit candidate sequences are valid for each read.

An alignment sequence may be determined by performing a multiple sequence alignment, MSA, between the hit candidate sequence, the read, and one or more sequences derived from the read. The multiple sequence alignment may be configured to align each of the hit candidate sequences, the read, and the one or more sequences derived from the read without introducing any gaps in the alignment sequence. This can help to find “perfect” alignments and therefore more meaningful results (i.e. less likely due to chance because of insertion of gaps) can be obtained in the consensus matrices.

The one or more sequences derived from the read may comprise at least one of: a trimmed read, wherein one or more letter codes of the read that each have a sequencing quality lower than a value specified by a received cut-off parameter are removed; and a base-called sequence, wherein positions of the read with an ambiguity letter code are replaced by a letter code for a primary base.

Each group of the plurality of groups of reads may comprise one or more forward reads of the respective target multivalent ISV for the group and one or more reverse reads of the respective target multivalent ISV.

Generating a set of candidate sequences of multivalent ISVs based on the received sequence information may comprise: receiving sequence information for each of one or more linkers; receiving an indication of a particular restriction enzyme recognition site; and generating the set of candidate sequences of multivalent ISVs using the sequencing information for the one or more linkers and the indication of the particular restriction enzyme recognition site.

The consensus matrix may comprise a score, at each position of the plurality of positions in the alignment sequence, for each primary base letter code out of a set of primary base letter codes. The assembly matrix may comprise, for each read in the group of reads, and for each position in the alignment sequence, either a letter code for a primary base, or an empty symbol indicating that no letter code for a primary base could be determined for the position of the read.

Each component ISV may be selected from a VL, a VH, a VHH, a humanized VHH and a camelized VH. Each of the component ISVs may be a monovalent ISV.

The sequence information for each target multivalent ISV may comprise a nucleic acid sequence. The sequence information for each component ISV may comprise a nucleic acid sequence. The nucleic acid sequences may be DNA sequences.

According to a further aspect of this specification, there is described an apparatus comprising one or more processors configured to perform the method of any one or more of the methods described herein.

According to a further aspect of this specification, there is described a computer-readable storage medium comprising instructions, which when executed by one or more processors, cause the one or more processors to perform the method of any one or more of the methods described herein.

### BRIEF DESCRIPTION OF THE FIGURES

So that the specification may be more easily understood, embodiments thereof will now be described by way of example only, with reference to the accompanying drawings in which:

FIG. 1 illustrates an example multivalent ISV.

FIG. 2 illustrates a flowchart of an example method for obtaining sequence information for multivalent ISVs.

FIG. 3 illustrates example portions of a consensus matrix for a target multivalent ISV.

FIG. 4 illustrates example portions of an assembly matrix for a target multivalent ISV.

FIG. 5 illustrates an example DNA sequence determined for each of two multivalent ISVs.

FIG. 6 illustrates an example amino acid sequence determined for each of two multivalent ISVs.

FIG. 7 is a schematic illustration of a system/apparatus for performing methods described herein.

### DETAILED DESCRIPTION

Various example implementations described herein relate to systems and methods for obtaining sequence information (e.g. nucleic acid sequences, such as DNA sequences) for multivalent ISVs. In particular, the described systems and methods generate a set of theoretical sequences for a library of multivalent ISVs, based on sequence information (e.g. DNA sequences) of component ISVs that form the multivalent ISVs. The theoretical sequences (which may also be referred to herein as theoretical constructs) are compared with sequencing results (or reads) obtained for fragments of a multivalent ISV, and one or more sequences derived from the sequencing results, in order to determine sequence information for the multivalent ISV. In this way, sequence information for ISVs is obtained in an automated and robust manner with a high level of accuracy.

A library of target multivalent ISVs is obtained from a plurality of component ISVs. The plurality of component ISVs may comprise one or more monovalent ISVs, one or more bivalent ISVs, one or more trivalent ISVs, or any other component ISV for which sequence information for the entire component ISV has been determined. In some embodiments, each of the component immunoglobulin single variable domains may be monovalent ISVs.

The library of target multivalent ISVs is created from the plurality of component ISVs using standard techniques. For example, the genomic DNA (or cDNA) of each ISV is extracted and purified, and subsequently digested utilising physical methods, or enzymatic methods, such as with a restriction enzyme, to create smaller double-stranded fragments. Adaptors (short, double-stranded pieces of synthetic DNA), are then ligated to the ends of these digested DNA fragments. Subsequently, the DNA library is clonally amplified to increase the signal detected from each target fragment during sequencing. During amplification, each DNA fragment in the library is bound to the surface of a bear or a flow-cell and can be amplified using PCR to create identical clones. This amplification creates clusters of DNA, each originating from a single library fragment, representing one of the plurality of component ISVs.

The DNA library of target multivalent ISVs is then sequenced using one of many sequencing methods well known to the skilled person in the art, including high-throughput next-generation sequencing (NGS) techniques, such as 454 Pyrosequencing, Ion Torrent semiconductor sequencing, sequencing by ligation (SOLID), or Illumina sequence. For example, the DNA fragments can be placed into a well along with DNA polymerases and primers that hybridise to the 3′ end of the template strand, and the complete complementary strand of each fragment is synthesised. As such, DNA sequences for the plurality of component ISVs and DNA sequences for one or more linkers that link together component ISVs (and/or common regions) can be obtained using a plurality of primers for sequencing including e.g. one or more forward primers and/or one or more reverse primers, and a restriction enzyme.

For example, the library of target multivalent ISVs may be sequenced using one or more microplates, wherein each well of a microplate corresponds to a different fragment of a multivalent ISV produced by using a different combination of the component ISVs and/or a different primer used for sequencing. As a particular example, a different 96-well plate may be used for each primer, and corresponding positions of different well-plates may correspond to a same clone. For example, well A01 in a first plate may correspond to a clone that is sequenced using a forward primer, and well A01 of a second plate may correspond to the same clone being sequenced using a reverse primer. Alternatively, a single plate may be sequenced using different primers, wherein each clone is sequenced by a particular primer of the various primers in a different well.

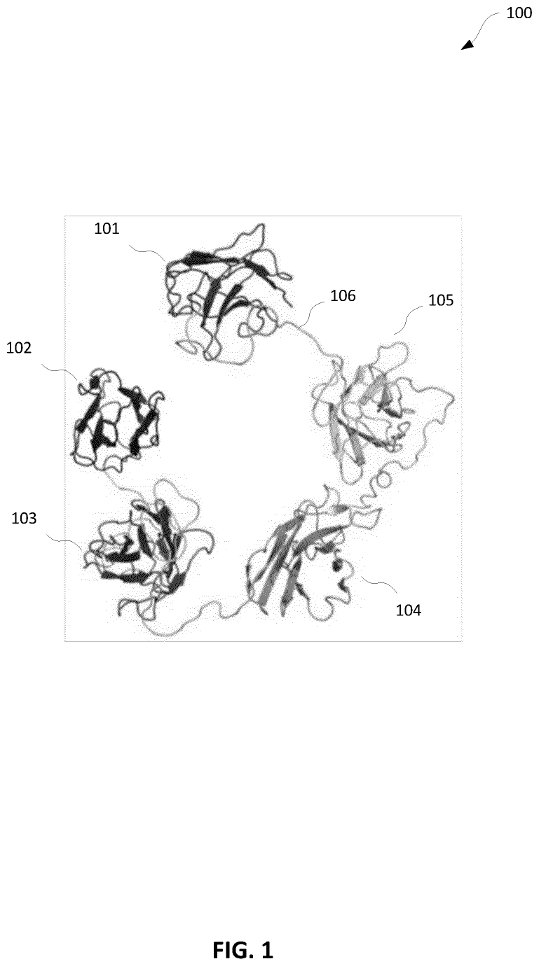

FIG. 1 illustrates an example target multivalent ISV 100. The multivalent ISV 100 shown in FIG. 1 is a pentavalent ISV, consisting of five monovalent ISVs 101, 102, 103, 104, 105, linked together by linkers 106. Each of the monovalent ISVs can be directed to different sites (i.e. antigens) of a same target, or towards different targets.

The term “immunoglobulin single variable domain” (ISV), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g. monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulfide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISV) can for example be a heavy chain ISV, such as a V_{H}, V_{HH}, including a camelized V_{H} or humanized V_{HH}. In one embodiment, it is a V_{HH}, including a camelized V_{H} or humanized V_{HH}. Heavy chain ISVs can be derived from a conventional four-chain antibody or from a heavy chain antibody.

For example, the immunoglobulin single variable domain may be a single domain antibody (or an amino acid sequence that is suitable for use as a single domain antibody), a “dAb” or dAb (or an amino acid sequence that is suitable for use as a dAb) or a Nanobody® ISV (as defined herein, and including but not limited to a V_{HH}); other single variable domains, or any suitable fragment of any one thereof.

In particular, the immunoglobulin single variable domain may be a Nanobody® ISV (such as a V_{HH}, including a humanized V_{HH} or camelized V_{H}) or a suitable fragment thereof. [Note: Nanobody® and Nanobodies® are registered trademarks of Ablynx N.V.]

“V_{HH} domains”, also known as V_{HH}S, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. Nature 363: 446-448, 1993). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'S, reference is made to the review article by Muyldermans (Reviews in Molecular Biotechnology 74: 277-302, 2001).

The generation of immunoglobulin sequences, such as VHHs, has been described extensively in various publications, among which WO 94/04678, Hamers-Casterman et al. 1993 and Muyldermans et al. 2001 (Reviews in Molecular Biotechnology 74: 277-302, 2001). In these methods, camelids are immunized with the target antigen in order to induce an immune response against said target antigen. The repertoire of VHHs obtained from said immunization is further screened for VHHs that bind the target antigen.

In these instances, the generation of antibodies requires purified antigen for immunization and/or screening. Antigens can be purified from natural sources, or in the course of recombinant production. Immunization and/or screening for immunoglobulin sequences can be performed using peptide fragments of such antigens.

Immunoglobulin sequences of different origins, comprising mouse, rat, rabbit, donkey, human and camelid immunoglobulin sequences can be sequenced in the method described herein. Also, fully human, humanized or chimeric sequences can be sequenced in the method described herein. For example, camelid immunoglobulin sequences and humanized camelid immunoglobulin sequences, or camelized domain antibodies, e.g. camelized dAb as described by Ward et al (see for example WO 94/04678 and Riechmann, Febs Lett., 339:285-290, 1994 and Prot. Eng., 9:531-537, 1996) can be sequenced in the method described herein. Moreover, the ISVs are fused forming a multivalent and/or multispecific construct (for multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221).

A “humanized V_{HH}” comprises an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the prior art (e.g. WO 2008/020079). Again, it should be noted that such humanized V_{HH}S can be obtained in any suitable manner known per se and thus are not strictly limited to polypeptides that have been obtained using a polypeptide that comprises a naturally occurring VHH domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a (camelid) heavy chain antibody. This can be performed in a manner known per se, which will be clear to the skilled person, for example on the basis of the description in the prior art (e.g. Davies and Riechman (1994 and 1996), supra). Such “camelizing” substitutions are inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). In one embodiment, the V_{H} sequence that is used as a starting material or starting point for generating or designing the camelized V_{H} is a V_{H} sequence from a mammal, such as the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-Sequence or V_{HH} sequence). In one particular aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISV sequence used in the methods described herein may contain one or more of hallmark residues (as defined herein), such that the ISV sequence is a Nanobody® ISV, such as e.g. a V_{HH}, including a humanized V_{HH} or camelized V_{H}. Non-limiting examples of (suitable combinations of) such framework sequences will become clear from the further disclosure herein.

The total number of amino acid residues in a V_{H} domain and a V_{HH} domain will usually be in the range of from 110 to 120, often between 112 and 115. It should however be noted that smaller and longer sequences may also be suitable for the purposes described herein.

However, it should be noted that the ISVs comprised in the multivalent ISV polypeptide that is sequenced in the present method is not limited as to the origin of the ISV sequence (or of the nucleotide sequence used to express it), nor as to the way that the ISV sequence or nucleotide sequence is (or has been) generated or obtained. Thus, the ISV sequences may be naturally occurring sequences (from any suitable species) or synthetic or semi-synthetic sequences. In a specific but non-limiting aspect, the ISV sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences (and in particular camelized V_{H} sequences), as well as ISVs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

Generally, Nanobody ISVs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a Nanobody® ISV can be defined as an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody® ISV can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody® ISV can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
one or more of the amino acid residues at positions 11, 37, 44, 45, 47, 83, 84, 103, 104 and 108 according to the Kabat numbering are chosen from the Hallmark residues mentioned in Table A below.

TABLE A
Hallmark Residues in Nanobody ® ISVs

| Position | Human V_{H}3 | Hallmark Residues |
|---|---|---|
| 11 | L, V; predominantly L | L, S, V, M, W, F, T, Q, E, A, R, G, |
| | | K, Y, N, P, I; preferably L |
| 37 | V, I, F; usually V | F⁽¹⁾, Y, V, L, A, H, S, I, W, C, N, |
| | | G, D, T, P, preferably F⁽¹⁾ or Y |
| 44⁽⁸⁾ | G | E⁽³⁾, Q⁽³⁾, G⁽²⁾, D, A, K, R, L, P, |
| | | S, V, H, T, N, W, M, I; |
| | | preferably G⁽²⁾, E⁽³⁾ or Q⁽³⁾; most |
| | | preferably G⁽²⁾ or Q⁽³⁾. |
| 45⁽⁸⁾ | L | L⁽²⁾, R⁽³⁾, P, H, F, G, Q, S, E, T, |
| | | Y, C, I, D, V; preferably L⁽²⁾ or R⁽³⁾ |
| 47⁽⁸⁾ | W, Y | F⁽¹⁾, L⁽¹⁾ or W⁽²⁾ G, I, S, A, V, M, |
| | | R, Y, E, P, T, C, H, K, Q, N, D; |
| | | preferably W⁽²⁾, L⁽¹⁾ or F⁽¹⁾ |
| 83 | R or K; usually R | R, K⁽⁵⁾, T, E⁽⁵⁾, Q, N, S, I, V, G, |
| | | M, L, A, D, Y, H; preferably K or R; |
| | | most preferably K |
| 84 | A, T, D; | P⁽⁵⁾, S, H, L, A, V, I, T, F, D, R, |
| | predominantly A | Y, N, Q, G, E; preferably P |
| 103 | W | W⁽⁴⁾, R⁽⁶⁾, G, S, K, A, M, Y, L, F, |
| | | T, N, V, Q, P⁽⁶⁾, E, C; preferably W |
| 104 | G | G, A, S, T, D, P, N, E, C, L; |
| | | preferably G |
| 108 | L, M or T; | Q, L⁽⁷⁾, R, P, E, K, S, T, M, A, H; |
| | predominantly L | preferably Q or L⁽⁷⁾ |

Notes:
In particular, but not exclusively, in combination with KERE or KQRE at positions 43-46.
Usually as GLEW at positions 44-47.
Usually as KERE or KQRE at positions 43-46, e.g. as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
With both GLEW at positions 44-47 and KERE or KQRE at positions 43-46.
Often as KP or EP at positions 83-84 of naturally occurring V_{HH} domains.
In particular, but not exclusively, in combination with GLEW at positions 44-47.
With the proviso that when positions 44-47 are GLEW, position 108 is always Q in (non-humanized) V_{HH} sequences that also contain a W at 103.
The GLEW group also contains GLEW-like sequences at positions 44-47, such as for example GVEW, EPEW, GLER, DQEW, DLEW, GIEW, ELEW, GPEW, EWLP, GPER, GLER and ELEW.

As used herein, a VHH is a heavy chain only antibody (HcAb), which is approximately 15 kDa in size, and is naturally produced in e.g. camelids (VHH, from camels, alpacas, dromedaries, and llamas), and cartilaginous fishes (VNAR, from sharks). A VHH corresponds to the variable region of a heavy chain antibody.

ISVs have advantages over conventional antibodies: they are about ten times smaller than IgG molecules, and as a consequence properly folded functional ISVs can be produced by in vitro expression while achieving high yield. Furthermore, ISVs are very stable, resistant to the action of proteases, and can readily be engineered into bi- or multivalent forms.

As used herein, the term “monovalent ISV” denotes a compound that comprises or essentially consists of a single ISV.

As used herein, the term “multivalent ISV” denotes a compound that combines two or more ISVs within a single molecule.

In general, the term “multivalent” indicates the presence of multiple ISVs in a polypeptide. In one embodiment, the polypeptide is “bivalent”, i.e., comprises or consists of two ISVs. In one embodiment, the polypeptide is “trivalent”, i.e., comprises or consists of three ISVs. In another embodiment, the polypeptide is “tetravalent”, i.e. comprises or consists of four ISVDs. The polypeptide sequenced in the method described herein can thus be “bivalent”, “trivalent”, “tetravalent”, “pentavalent”, “hexavalent”, “heptavalent”, “octavalent”, “nonavalent”, etc., i.e., the polypeptide comprises or consists of two, three, four, five, six, seven, eight, nine, etc., ISVs, respectively. In one embodiment the multivalent ISV polypeptide is trivalent. In another embodiment the multivalent ISV polypeptide is tetravalent. In still another embodiment, the multivalent ISV polypeptide is pentavalent.

In one embodiment, the multivalent ISV polypeptide can also be multispecific. The term “multispecific” refers to binding to multiple different target molecules (also referred to as antigens). The multivalent ISV polypeptide can thus be “bispecific”, “trispecific”, “tetraspecific”, etc., i.e., can bind to two, three, four, etc., different target molecules, respectively.

For example, the polypeptide may be bispecific-trivalent, such as a polypeptide comprising or consisting of three ISVs, wherein two ISVs bind to a first target and one ISV binds to a second target different from the first target. In another example, the polypeptide may be trispecific-tetravalent, such as a polypeptide comprising or consisting of four ISVs, wherein one ISV binds to a first target, two ISVs bind to a second target different from the first target and one ISV binds to a third target different from the first and the second target. In still another example, the polypeptide may be trispecific-pentavalent, such as a polypeptide comprising or consisting of five ISVs, wherein two ISVs bind to a first target, two ISVs bind to a second target different from the first target and one ISV binds to a third target different from the first and the second target.

In one embodiment, the multivalent ISV polypeptide can also be multiparatopic. The term “multiparatopic” refers to binding to multiple different epitopes on the same target molecules (also referred to as antigens). The multivalent ISV polypeptide can thus be “biparatopic”, “triparatopic”, etc., i.e., can bind to two, three, etc., different epitopes on the same target molecules, respectively.

As used herein, the term “linker” denotes a peptide that fuses together two or more (poly)peptides (e.g. ISVs, common regions as defined herein, etc.) into a single molecule. The use of linkers to connect two or more (poly)peptides is well known in the art. Further exemplary peptidic linkers are shown in Table A. One often used class of peptidic linker are known as the “Gly-Ser” or “GS” linkers. These are linkers that essentially consist of glycine (G) and serine(S) residues, and usually comprise one or more repeats of a peptide motif such as the GGGGS (SEQ ID NO:2) motif (for example, having the formula (Gly-Gly-Gly-Gly-Ser)ₙ in which n may be 1, 2, 3, 4, 5, 6, 7 or more). Some often-used examples of such GS linkers are 9GS linkers (GGGGSGGGS, SEQ ID NO: 5), 15GS linkers (n=3) and 35GS linkers (n=7). Reference is for example made to Chen et al., Adv. Drug Deliv. Rev. 2013 Oct. 15; 65(10): 1357-1369; and Klein et al., Protein Eng. Des. Sel. (2014) 27 (10): 325-330.

TABLE A
Linker sequences (“ID” refers to the
SEQ ID NO as used herein)

| Name | ID | Amino acid sequence |
|---|---|---|
| 3A linker | 1 | AAA |
| 5GS linker | 2 | GGGGS |
| 7GS linker | 3 | SGGSGGS |
| 8GS linker | 4 | GGGGSGGS |
| 9GS linker | 5 | GGGGSGGGS |
| 10GS linker | 6 | GGGGSGGGGS |
| 15GS linker | 7 | GGGGSGGGGSGGGGS |
| 18GS linker | 8 | GGGGSGGGGSGGGGSGGS |
| 20GS linker | 9 | GGGGSGGGGSGGGGSGGGGS |
| 25GS linker | 10 | GGGGSGGGGSGGGGSGGGGSGGGGS |
| 30GS linker | 11 | GGGGSGGGGSGGGGSGGGGSGGGGSGGGGS |
| 35GS linker | 12 | GGGGSGGGGSGGGGSGGGGSGGGGSGGGGSGGGGS |
| 40GS linker | 13 | GGGGSGGGGSGGGGSGGGGSGGGGSGGGGSGGGGSGGGGS |
| G1 hinge | 14 | EPKSCDKTHTCPPCP |
| 9GS-G1 hinge | 15 | GGGGSGGGSEPKSCDKTHTCPPCP |
| Llama upper long | 16 | EPKTPKPQPAAA |
| hinge region | | |
| G3 hinge | 17 | ELKTPLGDTTHTCPRCPEPKSCDTPPPCPRCPEPKSCDTPPPC |
| | | PRCPEPKSCDTPPPCPRCP |

As used herein, the term “common region” denotes a region that may be present within each of a plurality of target multivalent ISVs. A common region may comprise a V_{H}, a V_{L}, a cytokine or other protein/peptide, which may be attached to a linker. A common region may be used to extend the half-life of the multivalent ISV in vivo.

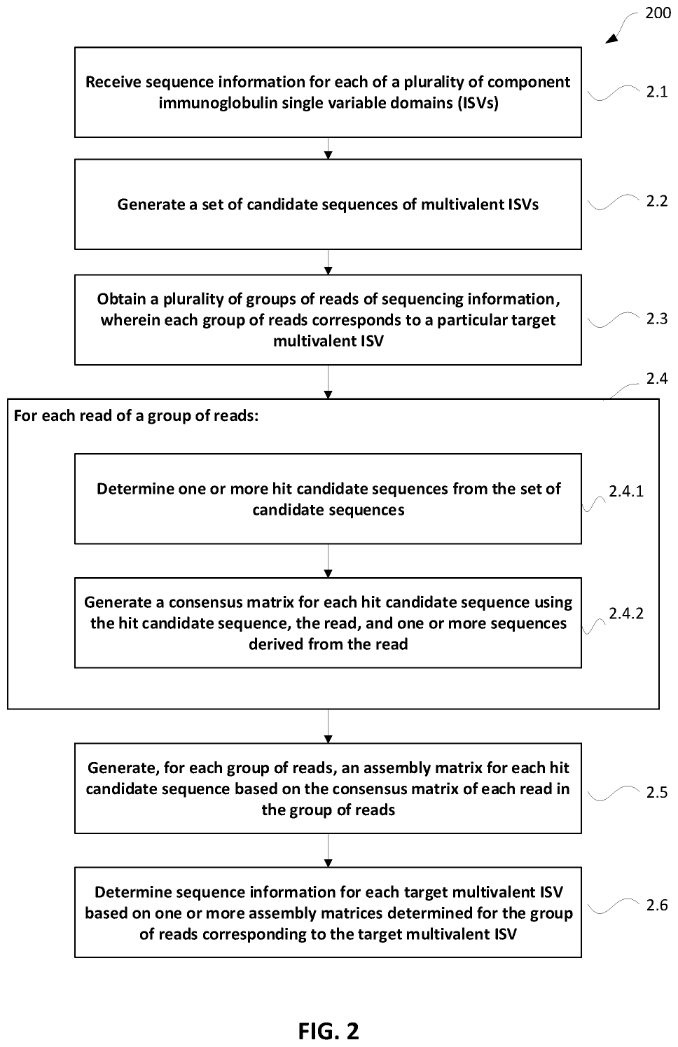

FIG. 2 illustrates a flowchart of an example method 200 for obtaining sequence information for multivalent ISVs. The method 200 produces sequence information for each target multivalent ISV in a library of target multivalent ISV, wherein the library of target multivalent ISVs was created from a plurality of component ISVs, as described previously.

In step 2.1, sequence information is received for each of a plurality of component ISVs. Each target multivalent ISV comprises a plurality of the plurality of component ISVs. The sequence information for each component ISV may be a nucleic acid sequence, such as a DNA sequence or an RNA sequence, or the sequence information may be an amino acid sequence. The sequence information may be provided in the form of a FASTA file, a raw data file (e.g. ABIF file format) or data stream derived from a sequencing device for each component ISV.

Further information for generating a set of candidate sequences for the library of target multivalent ISVs may also be received. For example, sequence information for each of one or more linkers may be received. Sequence information for each of one or more common regions may be received. The sequence information may be a nucleic acid sequence, such as a DNA sequence or an RNA sequence, or the sequence information may be an amino acid sequence. The sequence information may be provided in the form of a FASTA file for each linker. Sequence information for one or more flanking primers for each component ISV may also be received. Sequence information for one or more constant regions may also be received. An indication of a particular restriction enzyme recognition site used for cloning may also be received.

Thus, information may be received that reflects molecules and compounds used to generate the library of target multivalent ISVs using cloning techniques. The received information is used to generate a library of theoretical sequences of multivalent ISVs in silico.

In step 2.2, a set of candidate sequences of multivalent ISVs is generated based on the received sequence information. The set of candidate sequences is a set of all the theoretical sequences of multivalent ISVs (each such theoretical sequence also referred to as a theoretical construct) that can be created using the component ISVs, and where appropriate, linkers, and common regions. In some examples, the set of candidate sequences may be determined from a fixed set of component ISVs (e.g. just one ISV per position of a multivalent ISV) but different linkers to identify a best linker combination for a multivalent ISV comprising the fixed set of component ISVs. The set of candidate sequences is generated in a combinatorial manner, ensuring that every possible theoretical construct is reflected in the set of candidate sequences. The set of candidate sequences comprise sequence information for each of the theoretical constructs. The sequence information may be a nucleic acid sequence, such as a DNA sequence or an RNA sequence, or the sequence information may be an amino acid sequence. The sequence information of each theoretical construct may be stored (e.g. in the form of a FASTA file) or otherwise maintained in memory.

In step 2.3, a plurality of groups of reads of sequencing information are obtained. Each group of reads corresponds to a particular target multivalent ISV. Each group of reads comprises one or more forward reads of a particular target multivalent ISV and/or one or more reverse reads of the particular target multivalent ISV. Each read in a group of reads is obtained from sequencing fragments of the same target multivalent ISV using different primers. Forward reads are reads obtained using forward primers, and reverse reads are reads obtained using reverse primers. Any suitable combination of forward reads and/or reverse reads may be used to form the group of reads. For example, a group of reads may consist of two or more forward reads, a group of reads may consist of two or more reverse reads, a group of reads may consist of one or more forward reads and one or more reverse reads, etc. Each group of the plurality of groups of reads may comprise reads obtained from the same combination of forward primers and/or reverse primers, e.g. each group of the plurality of groups of reads may comprise the same number of forward and/or reverse reads.

A read is sequencing information for a fragment of a multivalent ISV, as obtained by a sequencing machine. A read comprises a letter code for each position of a plurality of positions of the read. Each letter code specifies either a letter code for a primary base or an ambiguity letter code (e.g. an IUPAC ambiguity letter code). Thus, the reads may lack base calls wherein a sequencing provider estimates primary base letter codes for ambiguous/low quality readings. Each read may also comprise (or otherwise be associated with) a sequencing quality for each position of the read. The sequencing quality measures a confidence in the prediction of the position's letter code.

A determination may be made that a read belongs in a particular group of reads corresponding to a particular target multivalent ISV based on metadata associated with the read. For example, the metadata may indicate a plate identifier, a sample identifier, and/or a well identifier, which identifiers may be used to group together reads corresponding to the same target multivalent ISV. For example, a read with an identifier for well C07 in a first plate may be grouped together with a read for well C07 in a second plate using the metadata associated with the reads that indicate a well identifier and a plate identifier.

Step 2.4 comprises steps 2.4.1 and 2.4.2 which are performed for each read of a group of reads. Further, the steps are repeated for each group of reads. Steps 2.4.1 and 2.4.2 (and subsequent steps) may be performed in parallel, e.g. by use of multi-core central processing unit (CPU) s. For example, each group of reads may be processed in the methods described below separately, with the processing of reads in the same group being performed on the same CPU-thread.

In step 2.4.1, one or more hit candidate sequences are determined from the set of candidate sequences. Each of the one or more hit candidate sequences comprises a matching portion with a corresponding portion of the read. The determination may be made using a pattern matching process that compares the read (or portions thereof) with each candidate sequence in the set of candidate sequences. Any suitable pattern matching process may be used, such as a Rabin-Karp algorithm, a Knuth-Morris-Pratt algorithm, a Boyer-Moore algorithm, etc.

The reads may first be pre-processed before the pattern-matching process is performed. A start position may be determined for the read, and letter codes of the read before the start position may be read. The start position may be predetermined and constant, e.g. the same start position may be used for every read. Trimming the beginning portion of reads may help to remove residues that are associated with the cloning process and which might not form part of sequence information for a multivalent ISV. A position of the read that first specifies an ambiguity letter code (e.g. an IUPAC ambiguity letter code) may be determined and letter codes of the read that have a position beginning from the determined position until an end position of the read may be removed. Removing letter codes in this way removes ambiguity letter codes from the read. A cutoff parameter may be received, indicating a desired level of quality for the processed reads. A different value for the cutoff parameter may be received for each read. The read may be trimmed, comprising removing one or more letter codes of the read that each have a sequencing quality lower than the value specified by the cutoff parameter.

The hit candidate sequences for a read may be determined using a number of iterations. For example, in a first iteration, a comparison may be made between the (pre-processed) read and each candidate sequence in the set of candidate sequences. A pattern-matching process is performed to determine whether the read is contained in any of the candidate sequences. Any candidate sequence comprising a portion that matches with the read may be added to the hit candidate sequences for the read. The number of hit candidate sequences may be limited to a maximum number of hit candidate sequences. If the read is not contained in any of the candidate sequences, the read may be trimmed by removing one or more letter codes from the end of the read to produce a shortened read for a subsequent iteration. In some embodiments, at each iteration a single letter code may be removed from the end of the read. Removing a smaller number of letter codes at each iteration may lead to greater accuracy in the determined sequence information for the target multivalent ISVs.

A comparison may be made between the shortened read of the iteration and each candidate sequence in the set of candidate sequences, for example by performing a pattern-matching process. If the shortened read of the iteration matches a particular candidate sequence, the particular candidate sequence may be added to the one or more hit candidate sequences. The previous steps may be repeated until one or more conditions are satisfied. For example, the steps may be repeated until the number of hit candidate sequences reaches a maximum number, and/or until the shortened read is shorter than a minimum length.

The hit candidate sequences for reads in a group may be pruned and hit candidate sequences may be determined for the whole group of reads. For example, the respective sets of hit candidate sequences that have been determined for each read in the group of reads may be intersected. In other words, each candidate sequence in the hit candidate sequences comprises a respective matching portion corresponding to each read in the group of reads. For example, if a particular candidate sequence comprises a matching portion for a forward read of a group but does not contain a matching portion for the corresponding reverse read of the group, then the particular candidate sequence may be removed from the hit candidate sequences.

In step 2.4.2, a consensus matrix (or any other suitable data format, e.g. a list of lists, a dictionary, etc.) is generated for each hit candidate sequence, using the hit candidate sequence, the read and one or more sequences derived from the read. In cases where reads were pre-processed in step 2.4.1, the term read here refers to the read prior to performing quality-based trimming (i.e. an untrimmed read). The consensus matrix specifies, for each position of a plurality of positions in an alignment sequence, a consensus between the hit candidate sequence, the read, and the one or more sequences derived from the read. The one or more sequences derived from the read comprise at least one of: a trimmed read, wherein one or more letter codes of the read that each have a sequencing quality lower than a value specified by a received cutoff parameter are removed; and a base-called sequence, wherein positions of the read with an ambiguity letter code are replaced by a letter code for a primary base. The base-called sequence may be determined in any appropriate manner.

The alignment sequence may be determined by performing a multiple sequence alignment (MSA) between the hit candidate sequence, the read, and one or more sequences derived from the read. The alignment sequence is a sequence that best, or sufficiently, aligns each of the sequences with each other. In some cases, the multiple sequence alignment may be configured to align each of the hit candidate sequence, the read, and the one or more sequences derived from the read without introducing any gaps in the alignment sequence. In these cases, the alignment sequence may be the same as the hit candidate sequence. Any suitable MSA technique may be used, such as techniques involving dynamic programming methods, iterative methods, hidden Markov models, multiple sequence comparison by log-expectation, etc.

The consensus matrix may comprise a score, at each position of the plurality of positions in the alignment sequence, for each primary base letter code out of a set of primary base letter codes. The score indicates how many sequences (i.e. those used to form the consensus matrix) agree on a particular base letter code for a particular position.

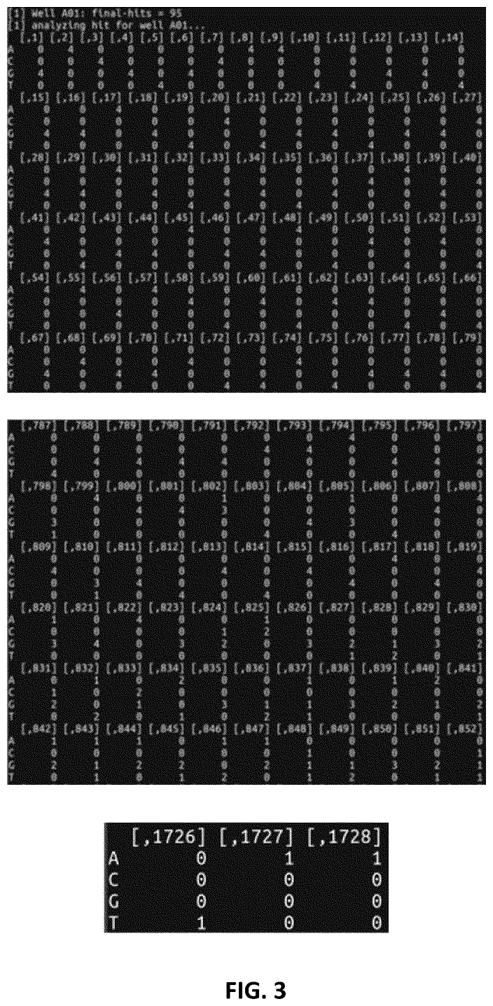

Turning briefly to FIG. 3, FIG. 3 illustrates example portions of a consensus matrix for an alignment sequence for a target multivalent ISV. The example consensus matrix displayed in FIG. 3 displays a consensus matrix determine for a forward read. Furthermore, FIGS. 3-6 illustrate examples of the methods and systems described herein where microplates were used to sequence the target multivalent ISVs. For example, each of these figures show aspects relating to a sequence for well A01, corresponding to a particular target multivalent ISV.

The columns of the consensus matrix are indexed by an index corresponding to the position in the alignment sequence, and the rows are indexed by letter codes for primary bases. The example consensus matrix of FIG. 3 was generated using a total of four sequences for the alignment sequence: a hit candidate sequence, an untrimmed forward read, and two sequences derived from the forward read: a trimmed forward read, and a base-called forward read. As a result, the maximum score that can be obtained for a letter code for a particular position is 4. As can be seen in FIG. 3, the maximum score is reached in positions in the alignment sequence up until position 801. Therefore, for these positions, each of the sequences relating to the forward read agree on a particular letter code for a primary base, indicating a high confidence for the alignment.

In contrast, positions 1726, 1727, 1728 of the alignment sequence only have a score of 1 for the highest-scoring letter code for these positions. This indicates that only the hit candidate sequence is present in the alignment sequence, and that the sequences relating to the forward read could not be used to validate the candidate sequence at these positions.

Where the highest-scoring letter code for a particular position is greater than 1 but less than the maximum score (e.g. positions 823, 824), this indicates a lower confidence for the alignment. In these cases, the highest-scoring letter code and the corresponding score may be used when generating an assembly matrix.

Returning to FIG. 2, in step 2.5, for each group of reads, an assembly matrix (or any other suitable data format, e.g. a list of lists, a dictionary, etc.) is generated for each hit candidate sequence based on the consensus matrix of each read in the group of reads. The results of the consensus matrices are merged to form the assembly matrix.

The assembly matrix may comprise, for each read in the group of reads, and for each position in the alignment sequence, either a letter code for a primary base, or an empty symbol indicating that no letter code for a primary base could be determined for the position of the read.

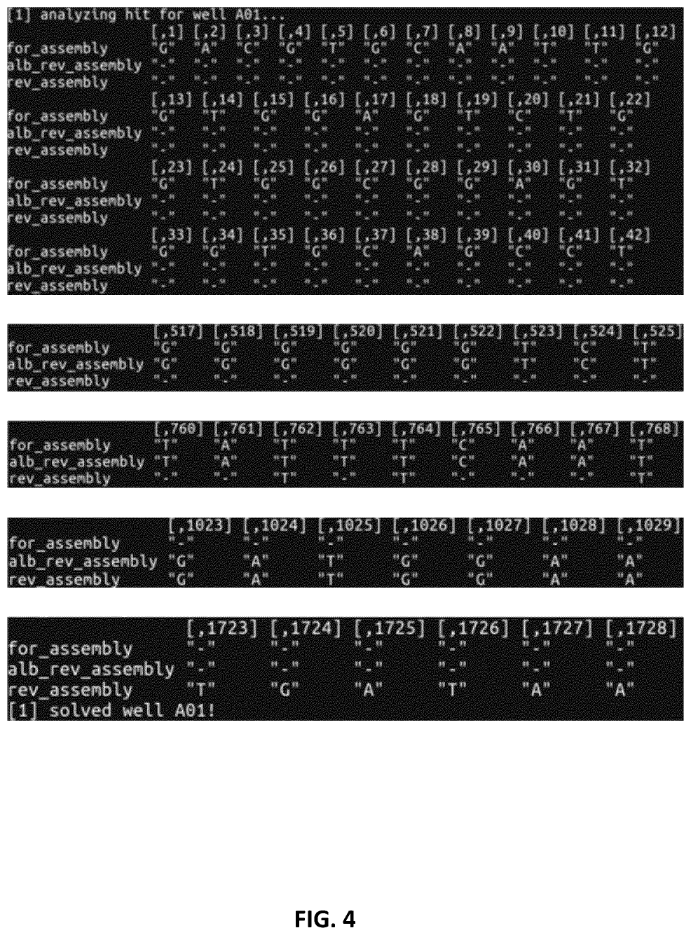

Turning briefly to FIG. 4, FIG. 4 illustrates example portions of an assembly matrix for an alignment sequence for a target multivalent ISV.

The columns of the assembly matrix are indexed by an index corresponding to the position in the alignment sequence, and the rows are indexed by the reads of the group of reads. The example assembly matrix shown in FIG. 4 merges together the results of consensus matrices determined from each of a forward read (denoted by “for_assembly”) and two reverse reads (denoted by “alb_rev_assembly” and “rev_assembly”) for a target multivalent ISV corresponding to well A01. It will be appreciated that the group of reads may comprise further reads (e.g. further forward and/or reverse reads), and/or may omit one of the forward/reverse reads.

Each of the entries of the assembly matrix are determined using the corresponding consensus matrix of the read associated with the entry. For example, the “for_assembly” entry of position 1 is determined using the consensus matrix of the forward read, which consensus matrix is shown in FIG. 3. The highest-scoring letter-code for the first position of this consensus matrix (which in this example is for the forward read) is the letter code “G”, and this highest score reaches the maximum score (4 in this example). As a result, letter code “G” is inserted into the entry of the first position of “for_assembly”. The other entries of the assembly matrix are determined in a similar manner, with the highest-scoring letter codes at each position of the consensus matrix of each read typically being entered into the corresponding entry of the assembly matrix. Where the highest-score of a particular position of a consensus matrix equals 1 (e.g. position 1726 in FIG. 3), an empty symbol (shown as a dash in FIG. 4) indicating that no letter code for a primary base could be determined for the position of the read is inserted instead (e.g. as shown in the entry for “for_assembly” in position 1726 in FIG. 4).

As shown in the example of FIG. 4, positions 517 to 525 of the alignment sequence are confirmed by both the forward read and the reverse read corresponding to “alb_rev_assembly”, indicating that it is likely that the hit candidate sequence corresponding to the assembly matrix is correct at these positions. Positions 762, and 764 are confirmed by all reads indicating a higher likelihood that the hit candidate sequence is correct at these positions.

Returning to FIG. 2, in step 2.6, sequence information is determined for each target multivalent ISV based on one or more assembly matrices determined for the group of reads corresponding to the target multivalent ISV. The sequence information may be a nucleic acid sequence, such as a DNA sequence or an RNA sequence, or the sequence information may be an amino acid sequence. The sequence information may be stored in the form of a FASTA file.

For each hit candidate sequence, an assembled sequence is determined based on the assembly matrix corresponding to the hit candidate sequence. The assembled sequence comprises, for each position of the plurality of positions of the alignment sequence, a letter code specifying either a letter code for a primary base or an ambiguity letter code (e.g. IUPAC ambiguity letter code). For example, if for a particular position of the assembly matrix, the entry of each read for the position specifies an empty symbol, an N may be determined for the position in the assembled sequence. In accordance with IUPAC ambiguity letter codes, this specifies that any of the primary bases are possible at this position of the alignment sequence.

If, for a particular position of the assembly matrix, the entry of each read specifies the same letter code of a particular primary base, then the letter code of the particular primary base is determined for the position in the assembled sequence. For example, for position 762 in FIG. 4, a “T” is shown for all entries of the assembly matrix. As a result, the 762^{nd} position of the assembled sequence is determined to be “T”.

For a position of the assembly matrix, multiple letter codes for primary bases may be specified. In this case, a score for each of the multiple letter codes may be obtained, and a highest-scoring letter code may be determined for the position in the assembled sequence. For example, consider a position of an assembly matrix that specifies “T” for a forward read, “A” for a reverse read, and “T” for a further reverse read. A score for the “T” for the forward read may be determined from the score, as specified in the consensus matrix for the forward read, for the letter code “T” for the position. Similarly, a score for the “A” for the reverse read, and a score for the “T” for the further reverse read may be determined. The scores obtained from the consensus matrices may be used to determine a score for the letter codes of the assembly matrix, e.g. the “T” score for the forward read may be added to the “T” score for the further reverse read. If the respective scores for the letter codes are equal, then an ambiguity letter code is determined for the position in the assembled sequence, based on the multiple letter codes for primary bases. In this example, an IUPAC ambiguity code of “W” (specifying “A” or “T”) may be determined for the position in the assembled sequence. Quality data of the sequencing results (e.g. Per-base quality values (PCON) and/or PHRED scores) can also be used to determine the letter code for a position in the assembly matrix. For example, if the quality data for a particular read indicates a high sequencing quality for a particular letter code, then this letter code may be determined to be the letter code for the position of the assembly matrix.

However, if one of the consensus matrices of a particular read specifies a maximum score (e.g. 4 in the example described in relation to FIG. 3) for a particular letter code at the position, then the letter code of the particular primary base is determined for the position in the assembled sequence.

In situations where there is only one hit candidate sequence, the assembled sequence corresponding to the hit candidate sequence is used to provide sequencing information for the target multivalent ISV. For example, the sequencing information may be the assembled sequence. Additionally or alternatively, the sequencing information may be derived from the assembled sequence, e.g. in the form of an amino acid sequence determined (i.e. translated) from the assembled sequence.

In situations where the number of hit candidate sequences is greater than one, each assembled sequence may be compared with its corresponding hit candidate sequence. First, a pattern matching process may be performed to determine whether the assembled sequence is the same as its corresponding hit candidate sequence. If a particular assembled sequence exactly matches its hit candidate sequence then this particular assembled sequence is selected to provide sequencing information for the target multivalent ISV. For example, the sequencing information may be the assembled sequence. Additionally or alternatively, the sequencing information may be derived from the assembled sequence, e.g. in the form of an amino acid sequence determined from the assembled sequence.

If none of the assembled sequences match its corresponding hit candidate sequence, then an assembled sequence that most closely matches its hit candidate sequence is selected to provide sequencing information for the target multivalent ISV. For example, sequence alignment techniques may be used to compare each of the assembled sequences with its corresponding hit candidate sequence. In particular, a global pairwise alignment may be performed e.g. by using dot-matrix methods, dynamic programming, and/or word methods. A score may be determined for how well aligned the assembled sequence and its corresponding hit candidate sequence are. The sequence alignment may be configured to perform the alignment without introducing any gaps in the alignment. The assembled sequence with the highest score may be selected to provide sequencing information for the target multivalent ISV.

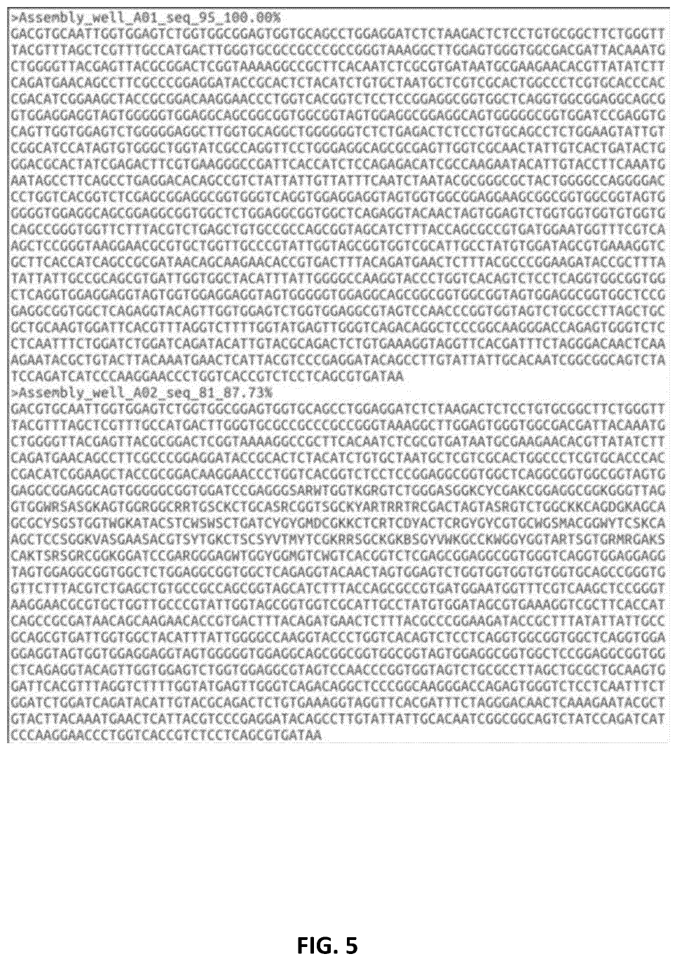

FIG. 5 illustrates an example DNA sequence determined for each of two multivalent ISVs. As shown in FIG. 5, the DNA sequence for the target multivalent ISV corresponding to well A01 was determined with a 100% match with its corresponding hit candidate sequence (seq_95 from the set of candidate sequences). For the target multivalent ISV corresponding to well A02, the DNA sequence that was determined shows 87.7% identity with the most closely matching hit candidate sequence (seq_81) from the set of candidate sequences. The DNA sequences may be stored in any appropriate format, e.g. in a FASTA file.

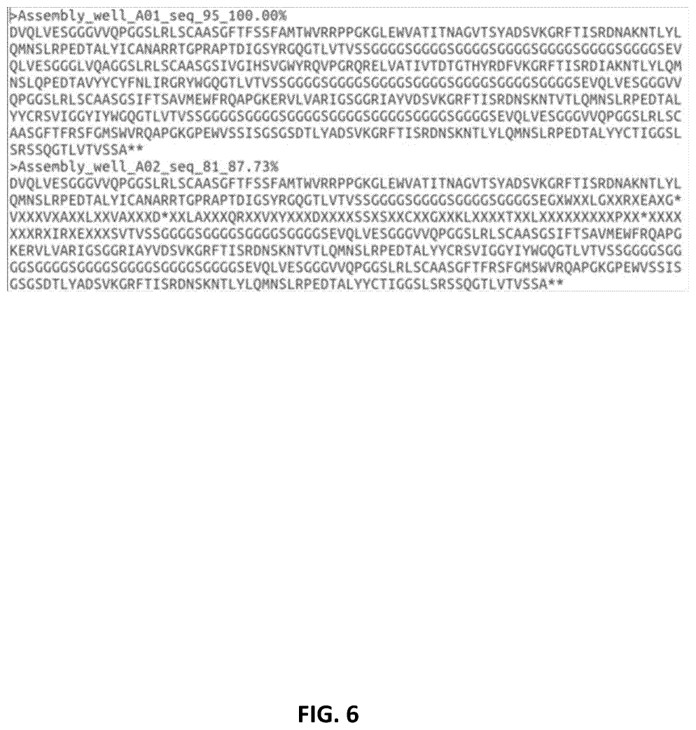

FIG. 6 illustrates an example amino acid sequence determined for each of two multivalent ISVs, corresponding to the DNA sequences illustrated in FIG. 5. The amino acid sequences may be stored in any appropriate format, e.g. in a FASTA file.

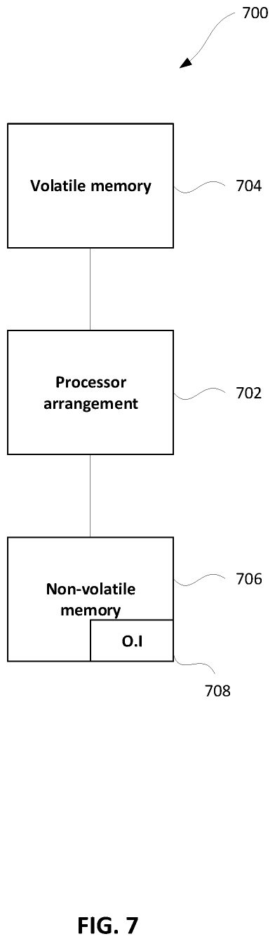

FIG. 7 is a schematic illustration of a system/apparatus for performing methods described herein. The system/apparatus shown is an example of a computing device. It will be appreciated by the skilled person that other types of computing devices/systems may alternatively be used to implement the methods described herein, such as a distributed computing system.

The apparatus (or system) 700 comprises one or more processors 702. The one or more processors control operation of other components of the system/apparatus 700. The one or more processors 702 may, for example, comprise a general purpose processor. The one or more processors 702 may be a single core device or a multiple core device. The one or more processors 702 may comprise a central processing unit (CPU) or a graphical processing unit (GPU). Alternatively, the one or more processors 702 may comprise specialised processing hardware, for instance a RISC processor or programmable hardware with embedded firmware. Multiple processors may be included.

The system/apparatus comprises a working or volatile memory 704. The one or more processors may access the volatile memory 704 in order to process data and may control the storage of data in memory. The volatile memory 704 may comprise RAM of any type, for example Static RAM (SRAM), Dynamic RAM (DRAM), or it may comprise Flash memory, such as an SD-Card.

The system/apparatus comprises a non-volatile memory 706. The non-volatile memory 706 stores a set of operation instructions 708 for controlling the operation of the processors 702 in the form of computer readable instructions. The non-volatile memory 706 may be a memory of any kind such as a Read Only Memory (ROM), a Flash memory or a magnetic drive memory.

The one or more processors 702 are configured to execute operating instructions 708 to cause the system/apparatus to perform any of the methods described herein. The operating instructions 708 may comprise code (i.e. drivers) relating to the hardware components of the system/apparatus 700, as well as code relating to the basic operation of the system/apparatus 700. Generally speaking, the one or more processors 702 execute one or more instructions of the operating instructions 708, which are stored permanently or semi-permanently in the non-volatile memory 706, using the volatile memory 704 to temporarily store data generated during execution of said operating instructions 708.

Implementations of the methods described herein may be realised as in digital electronic circuitry, integrated circuitry, specially designed ASICs (application specific integrated circuits), computer hardware, firmware, software, and/or combinations thereof. These may include computer program products (such as software stored on e.g. magnetic discs, optical disks, memory, Programmable Logic Devices) comprising computer readable instructions that, when executed by a computer, such as that described in relation to FIG. 7, cause the computer to perform one or more of the methods described herein.

Any system feature as described herein may also be provided as a method feature, and vice versa. As used herein, means plus function features may be expressed alternatively in terms of their corresponding structure. In particular, method aspects may be applied to system aspects, and vice versa.

Furthermore, any, some and/or all features in one aspect can be applied to any, some and/or all features in any other aspect, in any appropriate combination. It should also be appreciated that particular combinations of the various features described and defined in any aspects of the specification can be implemented and/or supplied and/or used independently.

Although several embodiments have been shown and described, it would be appreciated by those skilled in the art that changes may be made in these embodiments without departing from the principles of this disclosure, the scope of which is defined in the claims.

1-16. (canceled)

17. A computer-implemented method for obtaining sequence information for each of a plurality of target multivalent immunoglobulin single variable domains (ISVs), the method comprising:
receiving sequence information for each of a plurality of component ISVs, wherein each target multivalent immunoglobulin single variable domain (ISV) comprises a plurality of the component ISVs;
generating a set of candidate sequences of multivalent ISVs based on the received sequence information;
obtaining a plurality of groups of reads of sequencing information, wherein each group of reads corresponds to a particular target multivalent ISV of the plurality of target multivalent ISVs;
for each read of a group of reads:
determining one or more hit candidate sequences from the set of candidate sequences, wherein each of the one or more hit candidate sequences comprises a matching portion with a corresponding portion of the read, and
generating a consensus matrix for each hit candidate sequence using the hit candidate sequence, the read, and one or more sequences derived from the read, wherein the consensus matrix specifies, for each position of a plurality of positions in an alignment sequence, a consensus between the hit candidate sequence, the read, and the one or more sequences derived from the read;
generating, for each group of reads, an assembly matrix for each hit candidate sequence based on the consensus matrix of each read in the group of reads; and
determining sequence information for each target multivalent ISV based on one or more assembly matrices determined for the group of reads corresponding to the target multivalent ISV.

18. The method of claim 17, wherein a read comprises a letter code for each position of a plurality of positions of the read, each letter code specifying either a letter code for a primary base or an ambiguity letter code, and wherein determining, for a read, one or more hit candidate sequences from the set of candidate sequences comprises:
removing one or more letter codes from an end of the read to produce a shortened read for each iteration of a plurality of iterations;
performing a pattern matching process between the shortened read of an iteration and each candidate sequence; and
when a shortened read of an iteration matches a particular candidate sequence, adding the particular candidate sequence to the one or more hit candidate sequences.

19. The method of claim 17, wherein a read comprises a letter code for each position of a plurality of positions of the read, each letter code specifying either a letter code for a primary base or an ambiguity letter code, wherein the read specifies a sequencing quality for each position, and wherein determining, for a read, one or more hit candidate sequences from the set of candidate sequences comprises:
receiving a cutoff parameter;
determining a trimmed read, comprising removing one or more letter codes of the read that each have a sequencing quality lower than a value specified by the cutoff parameter;
determining a start position for the read, and removing letter codes of the read before the start position;
determining a position of the read that first specifies an ambiguity letter code; and
removing letter codes of the read that have a position beginning from the determined position until an end position of the read.

20. The method claim 17, wherein each candidate sequence in the hit candidate sequences comprises a respective matching portion corresponding to each read in the group of reads.

21. The method claim 17, wherein the alignment sequence is determined by performing a multiple sequence alignment, MSA, between the hit candidate sequence, the read, and one or more sequences derived from the read.

22. The method of claim 21, wherein the multiple sequence alignment is configured to align each of the hit candidate sequence, the read, and the one or more sequences derived from the read without introducing any gaps in the alignment sequence.

23. The method claim 17, wherein the one or more sequences derived from the read comprise at least one of:
a trimmed read, wherein one or more letter codes of the read that each have a sequencing quality lower than a value specified by a received cutoff parameter are removed; and
a base-called sequence, wherein positions of the read with an ambiguity letter code are replaced by a letter code for a primary base.

24. The method claim 17, wherein each group of the plurality of groups of reads comprises one or more forward reads of the respective target multivalent ISV for the group and one or more reverse reads of the respective target multivalent ISV.

25. The method claim 17, wherein generating a set of candidate sequences of multivalent ISVs based on the received sequence information comprises:
receiving sequence information for each of one or more linkers;
receiving an indication of a particular restriction enzyme recognition site; and
generating the set of candidate sequences of multivalent ISVs using the sequencing information for the one or more linkers and the indication of the particular restriction enzyme recognition site.

26. The method claim 17, wherein the consensus matrix comprises a score, at each position of the plurality of positions in the alignment sequence, for each primary base letter code out of a set of primary base letter codes.

27. The method of claim 26, wherein the assembly matrix comprises, for each read in the group of reads, and for each position in the alignment sequence, either a letter code for a primary base, or an empty symbol indicating that no letter code for a primary base could be determined for the position of the read.

28. The method claim 17, wherein each component ISV is selected from a VL, a VH, a VHH, a humanized VHH and a camelized VH, and optionally, wherein each of the component ISVs is a monovalent ISV.

29. The method claim 17, wherein the sequence information for each target multivalent ISV comprises a nucleic acid sequence, and/or the sequence information for each component ISV comprises a nucleic acid sequence, and optionally, wherein the nucleic acid sequence is a DNA sequence.

30. An apparatus comprising one or more processors configured to perform operations for obtaining sequence information for each of a plurality of target multivalent immunoglobulin single variable domains (ISVs), the operations comprising:
receiving sequence information for each of a plurality of component ISVs, wherein each target multivalent immunoglobulin single variable domain (ISV) comprises a plurality of the component ISVs;
generating a set of candidate sequences of multivalent ISVs based on the received sequence information;
obtaining a plurality of groups of reads of sequencing information, wherein each group of reads corresponds to a particular target multivalent ISV of the plurality of target multivalent ISVs;
for each read of a group of reads:
determining one or more hit candidate sequences from the set of candidate sequences, wherein each of the one or more hit candidate sequences comprises a matching portion with a corresponding portion of the read, and
generating a consensus matrix for each hit candidate sequence using the hit candidate sequence, the read, and one or more sequences derived from the read, wherein the consensus matrix specifies, for each position of a plurality of positions in an alignment sequence,

31. The apparatus of claim 30, wherein a read comprises a letter code for each position of a plurality of positions of the read, each letter code specifying either a letter code for a primary base or an ambiguity letter code, and wherein determining, for a read, one or more hit candidate sequences from the set of candidate sequences comprises:
removing one or more letter codes from an end of the read to produce a shortened read for each iteration of a plurality of iterations;
performing a pattern matching process between the shortened read of an iteration and each candidate sequence; and
when a shortened read of an iteration matches a particular candidate sequence, adding the particular candidate sequence to the one or more hit candidate sequences.

32. The apparatus of claim 30, wherein a read comprises a letter code for each position of a plurality of positions of the read, each letter code specifying either a letter code for a primary base or an ambiguity letter code, wherein the read specifies a sequencing quality for each position, and wherein determining, for a read, one or more hit candidate sequences from the set of candidate sequences comprises:
receiving a cutoff parameter;
determining a trimmed read, comprising removing one or more letter codes of the read that each have a sequencing quality lower than a value specified by the cutoff parameter;
determining a start position for the read, and removing letter codes of the read before the start position;
determining a position of the read that first specifies an ambiguity letter code; and
removing letter codes of the read that have a position beginning from the determined position until an end position of the read.

33. The apparatus of claim 30, wherein each candidate sequence in the hit candidate sequences comprises a respective matching portion corresponding to each read in the group of reads.

34. The apparatus of claim 30, wherein the alignment sequence is determined by performing a multiple sequence alignment, MSA, between the hit candidate sequence, the read, and one or more sequences derived from the read.

35. A computer-readable storage medium comprising instructions, which when executed by one or more processors, cause the one or more processors to perform operations for obtaining sequence information for each of a plurality of target multivalent immunoglobulin single variable domains (ISVs), the operations comprising:
receiving sequence information for each of a plurality of component ISVs, wherein each target multivalent immunoglobulin single variable domain (ISV) comprises a plurality of the component ISVs;
generating a set of candidate sequences of multivalent ISVs based on the received sequence information;
obtaining a plurality of groups of reads of sequencing information, wherein each group of reads corresponds to a particular target multivalent ISV of the plurality of target multivalent ISVs;
for each read of a group of reads:
determining one or more hit candidate sequences from the set of candidate sequences, wherein each of the one or more hit candidate sequences comprises a matching portion with a corresponding portion of the read, and
generating a consensus matrix for each hit candidate sequence using the hit candidate sequence, the read, and one or more sequences derived from the read, wherein the consensus matrix specifies, for each position of a plurality of positions in an alignment sequence, a consensus between the hit candidate sequence, the read, and the one or more sequences derived from the read;
generating, for each group of reads, an assembly matrix for each hit candidate sequence based on the consensus matrix of each read in the group of reads; and
determining sequence information for each target multivalent ISV based on one or more assembly matrices determined for the group of reads corresponding to the target multivalent ISV.

36. The non-transitory computer-readable storage medium of claim 35, wherein a read comprises a letter code for each position of a plurality of positions of the read, each letter code specifying either a letter code for a primary base or an ambiguity letter code, and wherein determining, for a read, one or more hit candidate sequences from the set of candidate sequences comprises:
removing one or more letter codes from an end of the read to produce a shortened read for each iteration of a plurality of iterations;
performing a pattern matching process between the shortened read of an iteration and each candidate sequence; and
when a shortened read of an iteration matches a particular candidate sequence, adding the particular candidate sequence to the one or more hit candidate sequences.
